(19) United States
(12) Patent Application Publication
(10) Pub. No.: US 2018/0040139 A1
(43) Pub. Date: Feb. 8, 2018

(54) METHOD FOR DETERMINING THE OFFSET BETWEEN THE CENTRAL AND OPTICAL AXES OF AN ENDOSCOPE

(71) Applicants: INSTITUT DE RECHERCHE SUR LES CANCERS DE L'APPAREIL DIGESTIF - IRCAD, STRASBOURG (FR); INSTITUT HOSPITALO-UNIVERSITAIRE DE CHIRURGIE MINI-INVASIVE GUIDEE PAR L'IMAGE, STRASBOURG (FR)

(72) Inventors: Sylvain BERNHARDT, Strasbourg (FR); Vincent AGNUS, Illkirch-Graffenstaden (FR); Stephane NICOLAU, Kehl (DE)

(21) Appl. No.: 15/548,359

(22) PCT Filed: Feb. 1, 2016
(86) PCT No.: PCT/FR2016/050203

§ 371 Date: Aug. 2, 2017

(30) Foreign Application Priority Data
Feb. 2, 2015 (FR) 1550792

(51) Int. Cl. G06T 7/73; G06T 7/33; G06K 9/46; G06T 7/246; G06T 7/529; G06T 7/174; A61B 1/00; G06T 7/00

(52) U.S. Cl. CPC G06T 7/73; A61B 1/00009; G06T 7/33; G06T 7/0012; G06T 7/248; G06T 7/529; G06T 7/174; G06K 9/4633; G06K 2009/2045

(57) ABSTRACT

Disclosed is a method for determining the offset or misalignment between the central or rotational axis and the optical axis of a rigid endoscope or a similar imaging device including a rigid body having an outer casing cylindrically-shaped in the direction of the optical axis, or including at least one segment having a rigid end with such a casing. The method includes taking a plurality of images with a field of view limited by a contour, the positioning of which relative to the central axis is, for each image, physically defined and specific, a relative angular rotation between the contour and the endoscope taking place between two successive images, and of determining a point or a pixel in the successively acquired images whose position remains unchanged, the point corresponding to the projection in the image plane of the central or rotational axis of the rigid body of the endoscope.

The present invention concerns the calibration or adjustment of optical systems, and especially endoscopic devices, especially in the context of mini-invasive surgery.

More specifically, the purpose of the invention is to create a process for determining the offset or misalignment between the median axis and the optical axis of a rigid endoscope, or one consisting of at least a rigid end segment, as well as a mini-invasive investigative and/or surgical procedure.

In numerous endoscopic devices, the lenses and CCD sensor of which the camera consists are misaligned with the physical central axis (median axis) of the body of the endoscope. In other words, the optical axis is displaced in relation to the axis of rotation, either intentionally (as a result of the structure of the endoscope) or otherwise (due to a distortion of the endoscope through repeated use, a manufacturing defect or uncertainty in the manufacturing process).

This misalignment or shift between the two axes is problematic in the context of using such an endoscope in a hybrid operating theater in which intraoperative three-dimensional (3D) images (for example, those acquired during the intervention by means of CT scanner-type equipment with a rotational arm in C) and endoscopic images are used simultaneously, and even superimposed. With this aim, it is necessary to determine the spatial position of the endoscopic camera in relation to the intraoperative 3D image.

The classic approach in this situation of a person well-versed in the art, such as that disclosed in [Feuerstein, M., Mussack, T., Heining, S. M., & Navab, N. (2008). “Intraoperative laparoscope augmentation for port placement and resection planning in minimally invasive liver resection” in Medical Imaging, IEEE Transactions 27(3), 355-369], consists of introducing an optical placement system into the operating room, fixing optical markers on the camera and scanner, calibrating the position of the first marker in relation to the camera's optical center, calibrating the position of the second marker in relation to the scanner's mark, and creating conditions that will make it possible to view both markers simultaneously during the surgical intervention. These stages are difficult to perform, the accuracy of the calibration obtained making it possible to achieve precision positioning of the camera in the order of 1 mm, using the leverage effect (since the body of an endoscope is often longer than 30 cm), corresponding to an error of at least 3 mm from the nominal distance of the shot.

The inventors recently proposed and assessed an approach that would make it possible to avoid introducing an additional optical positioning system consisting of positioning the body of the endoscope facing the area of interest and acquiring the scanned image in such a way that the end of the endoscope appears in the 3D image [S. Bernhardt, S. A. Nicolau, V. Agnus, L. Soler, C. Doignon, J. Marescaux. “Automatic Detection of Endoscope in Intraoperative CT Image: Application to AR Guidance in Laparoscopic Surgery”, in IEEE International Symposium on Biomedical Imaging (ISBI 2014), pp 563-567]. The end and orientation of the endoscope can then be automatically located in the 3D images and a virtual camera is created with a view of the area of interest that is identical to that of the actual endoscope, so as to be able to “increase” the endoscopic vision using 3D intraoperative data. Nevertheless, the expected accuracy could not be validated with larger quantities after the performance of encouraging preliminary tests, due to lack of a guarantee of superimposition of the central and optical axes in the endoscopes used.

The inventors deduced that, in order to obtain a more accurate superimposition of the 3D images from those supplied by the endoscope, not only was prior determination of the intrinsic and extrinsic endoscopic camera settings (as would be known to a person well-versed in the art) required, but it would also be necessary to determine the reciprocal offsetting/misalignment of the endoscope's optical and median axes.

In particular, though not restrictively, in relation to the abovementioned context, the main purpose of the invention is to propose a simple, swift, and accurate solution for determining this last setting.

The purpose of the invention is thus a process for determining the offset or misalignment between the central or rotating axis and the optical axis of a rigid endoscope or similar camera device consisting of a rigid body with an external cylindrical casing profiled in the direction of the optical axis or consisting of at least one rigid end segment clad with such a casing,

a procedure characterized by the fact that it consists of taking a number of shots, using a camera or sensor that is part of the endoscope or similar device, with a field of vision limited by a contour that is polygonal, circular, or elliptical in shape, the positioning in relation to the central or rotating axis being defined physically and being specific to each shot, with a relative angular rotation between the contour and the endoscope or similar rotation between two successive shots. A point or pixel needs to be determined in images acquired successively, its position remaining unchanged between the various shots, with the point or pixel corresponding to the projection in the image plane of the central axis or rotation of the rigid body of the endoscope or similar instrument or of the rigid segment at the end.

The invention will be better understood through the following description, concerning the preferred methods of achieving the purpose. These are given as unrestrictive examples and explained with reference to the schematic drawings enclosed, in which:

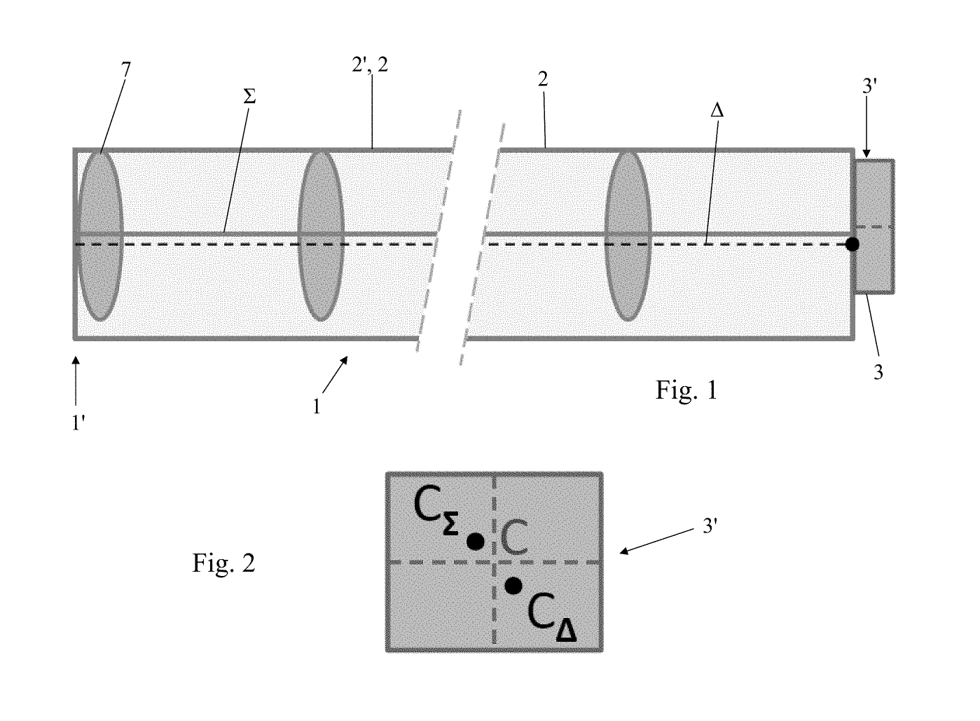

FIG. 1 is a partial schematic view in side elevation of a rigid endoscope with its camera;

FIG. 2 is a frontal elevation view of the image plane of the camera in FIG. 1, with an indication of the projections of the optical axes and rotation of the endoscope in FIG. 1;

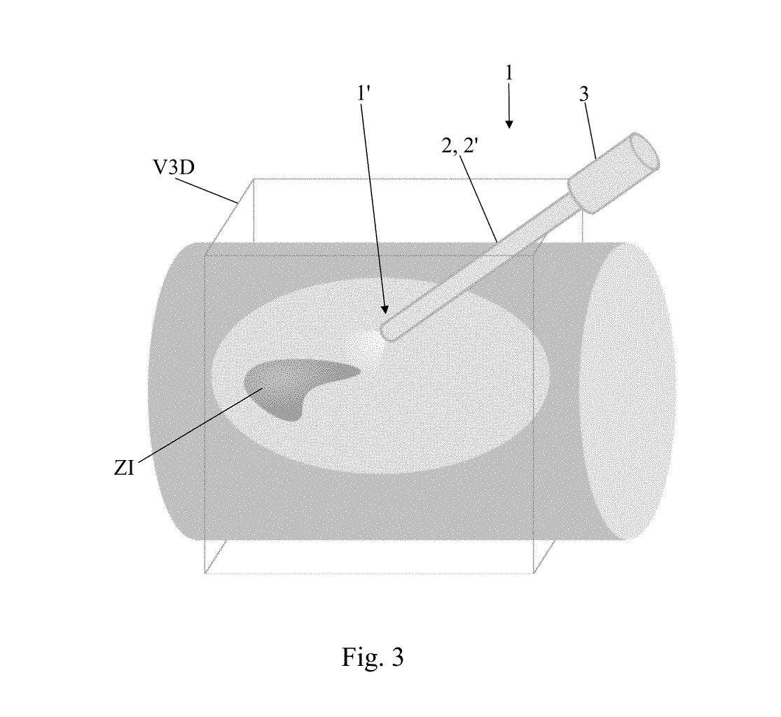

FIG. 3 is a partial schematic view of the body of a subject in which the endoscope representing FIG. 1 has been introduced, the V3D acquisition volume of the concomitant 3D imaging system also being indicated;

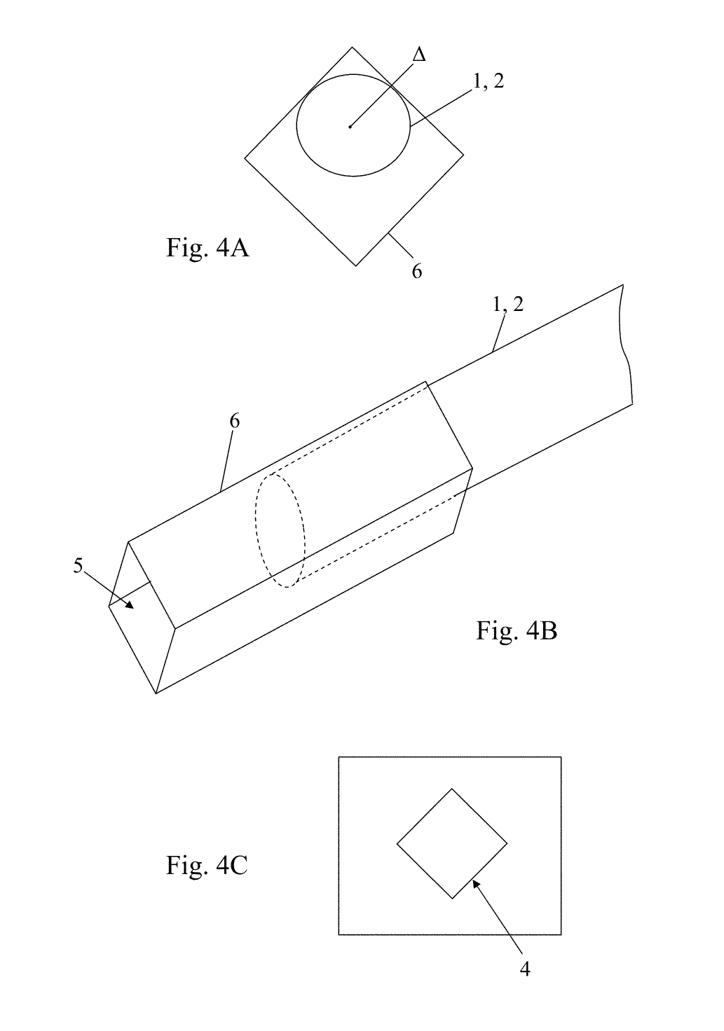

FIGS. 4A, 4B and 4C are respectively partial schematic views of the endoscope in FIG. 1 when fitted with a tubular part that is square in section, defining a contour that restricts the field of vision (FIGS. 4A and 4B) and the representation of the resulting image for the camera (FIG. 4C);

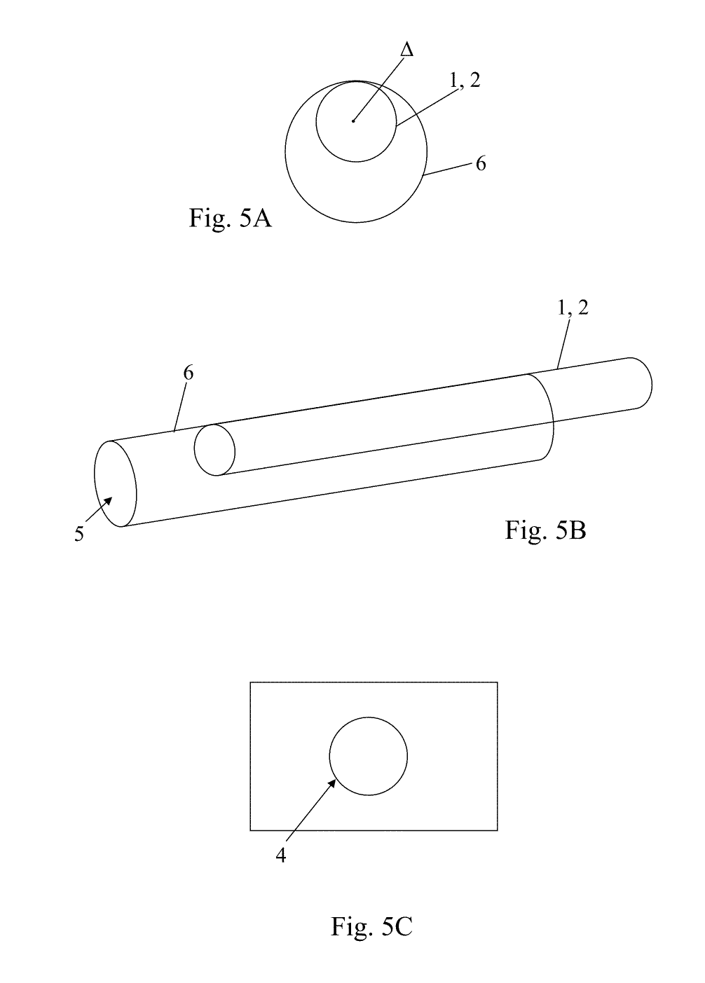

FIGS. 5A, 5B and 5C are partial schematic views respectively of the endoscope in FIG. 1 fitted with a tubular part that is circular in section, defining a contour restricting the field of vision (FIGS. 5A and 5B) and a representation of the resulting image for the camera (FIG. 5C);

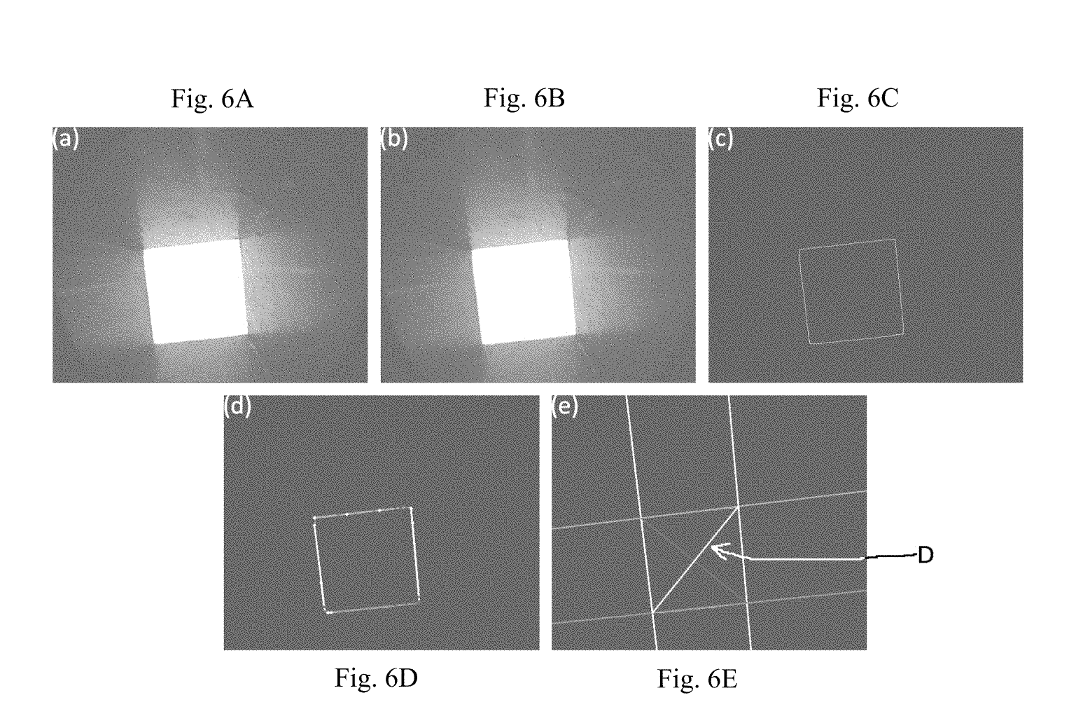

FIGS. 6A through 6E are an illustration of the manner in which the invention shown in the FIG. 4 can be implemented, with the various successive treatment operations for each image in order to be able to identify the diagonals;

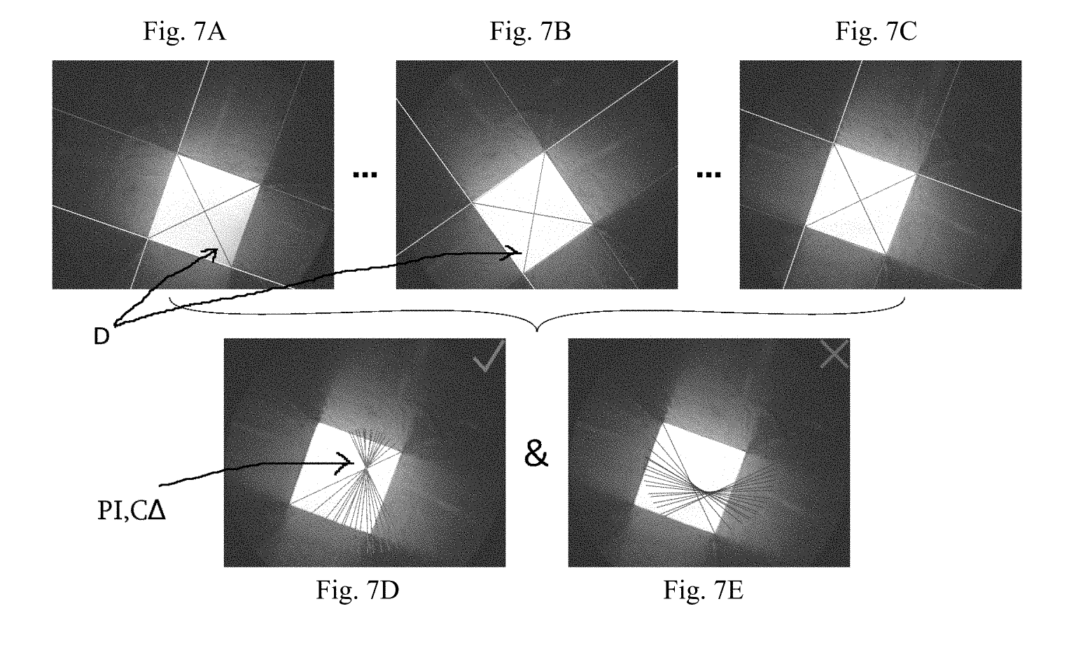

FIGS. 7A through 7E illustrate respectively, on the one hand, three examples of individual processed images that were obtained by implementing the processing operations illustrated in FIGS. 6A through 6E, for the three different relative angular positions between the square patch inserted and the endoscope (FIGS. 7A through 7C—mounting of FIG. 4), as well as the two cumulative images obtained by superimposing the two types of diagonals identified in the various individual images (FIGS. 7D and 7E), and,

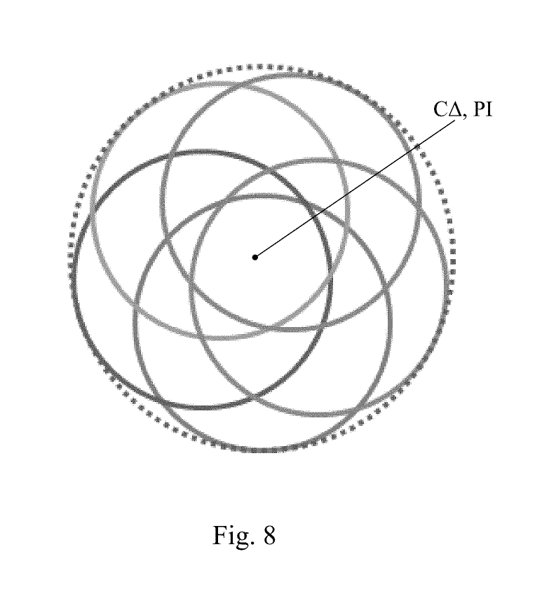

FIG. 8 is a representation of the cumulative images obtained by means of variable angle positions between an inserted patch that is circular in section and the endoscope (FIG. 5 assembly).

FIGS. 4 through 7 illustrate, with reference to the two construction variants that can be implemented, a process for determining the offsetting or misalignment between the central or rotating axis Δ and the optical axis Σ of a rigid endoscope 1 or similar camera-type device consisting of a rigid body 2 with an outer rigid cylindrical casing 2′ profiled in the direction of the optical (or median) axis, or consisting of at least one rigid segment at the end having such casing (close to the free end 1′ of the endoscope 1).

On the representations of FIGS. 1 and 2, note that the rotating axes Δ and optical axes Σ may not only be misaligned between each other (see the shift between their respective projections CΔ and CΣ in the image plane of camera 3), but even in relation to the center of the image plane—the rectangular window in FIG. 2—corresponding to point C of FIG. 2 (determining the misalignment between C and CΣ constitutes part of the calibration of the intrinsic settings of an endoscopic camera).

In accordance with the invention, this process consists of taking, with a camera or similar sensor 3 that is part of the endoscope or similar device 1, a multiplicity of shots with a field of vision limited by a contour 4 that is polygonal, circular or elliptical in shape, whose position in relation to the median or rotating axis Δ is physically defined and specific, for each shot, as having a relative angular rotation between the contour 4 and the endoscope or similar 1 intervening between two successive shots, and determining a point or a pixel PI, CΔ in the successively acquired images whose position remains unchanged between the various shots, the point or pixel PI, CΔ corresponding to the projection in the image plane 3 of the median axis of rotation Δ of the rigid body 2 of the endoscope 1 or similar or the rigid segment at the end of the endoscope.

This point PI is invariable in the image plane, regardless of the relative angular position between the contour 4 and the body 2 of the endoscope 1 (around the rotational axis Δ of the latter) and is formed by a limited number of pixels, preferably by a single pixel, corresponding to the orthogonal projection CΔ of the said axis of rotation Δ in the said image plane.

Preferably, and in order to facilitate the processing and identification of significant elements in the shots, it is planned that, in the case of images acquired successively, the contour 4 presents with a significant contrast in relation to the scene in the viewfinder, for example in terms of the various levels of grays, the differences in color, the differences in brightness, and the difference in the level of color saturation or similar mode.

In accordance with the practical implementation of a simple process, it is also possible to allow for the said contour to be defined by an aperture or cut-out 5 of a patch 6, associated temporarily with the endoscope 1 when various different shots are taken.

More specifically, and as shown in FIGS. 4 and 5 of the attached drawings, the aperture or patch 5 that restrictively defines contour 4 of the endoscope's field of vision or that of a similar device 1 can be supplied by a part 6, mounted temporarily on the endoscope 1, a similar device or a segment at the end of at least one of these, resting on the cylindrical outer casing 2′ for direct or indirect support.

In practice, before taking a series of shots, the insertion would then consist of inserting a tubular part or body 6, whose internal section is larger than the external section of the cylindrical casing 2′ and that could advantageously be fitted with a non-reflecting interior surface that is dark in color on the free end 1′ of the endoscope or similar 1, in such a way that it rests lengthwise on the latter's cylindrical body 2 or its rigid end and extends beyond the free end 1′ to define a restrictive window for the shot, with a field of vision that is limited peripherally by a contour 4, the angular positioning then being relatively modified between the said tubular body 6 and the cylindrical body 2 around the said median or rotating axis Δ between two successive shots.

In connection with the construction in the FIG. 4, and in accordance with the first method of implementing the procedure from which various operating stages emerge from the images shown in FIGS. 6 and 7, it could be planned that, in the case of a contour 4 provided by an polygonal opening or patch 5, preferably rectangular or square, the determination of the PI point, CΔ could remain fixed in the various shots and, while corresponding to the projection of the central or rotating axis Δ in the camera plane or similar 3, would consist of extraction of at least one diagonal D or line bisecting each of the scenes shown in the images resulting from successive shots, possibly after these have been processed, and determining at least approximately the shared PI intersection point for the various D diagonals or bisecting lines.

According to a characteristic mentioned above, the process can consist of applying different digital processing to each of the successive images so as to be able to remove at least the corner angles and even the major part or the entirety of the polygonal contour 4 that is visible in the various images, photographed from a variety of angles of the polygonal aperture 5, and in each image processed determining the diagonal D or the bisecting line of which one end touches the edge of contour 4 that is visible in the image in question, while superimposing the various images processed with their respective diagonal D or bisecting line selected and to determine, at least approximately, the intersection point PI shared by all the diagonals D or superimposed bisecting lines, for which the displacement between successive images has been mapped.

As an example of preliminary processing before using the images resulting from the succession of various shots, the process may consist, for each image acquired (FIG. 6A), of the performance of the following succession of processing operations: Bilateral filtering designed to eliminate noise while preserving the edges of contour 4 that are visible in the image in question (FIG. 6B); application of the Canny Edge Detector (FIG. 6C); application of the Hough Transform; grouping of the clearest segments extracted by direction and location (FIG. 6D); averaging each group of segments to define the angles, such as right angles, of each contour 4 visible on the various images acquired and defining a diagonal D or corresponding bisecting line (FIG. 6E); determining the intersection point PI, CΔ, at least approximately, of the diagonals D or bisecting lines selected from the various images (FIG. 7D).

In the case of a square contour 4, the abovementioned operations provide two sets of diagonals D in the various images, a single set making it possible to provide point CA.

In order to choose the right set of diagonals D (see FIG. 7), and more generally for determining point PI, CΔ of the intersection, at least approximately, for the diagonals D or bisecting lines chosen from the images processed resulting from various shots taken, the process may consist of applying the least squares method and defining them by calculating the position of point PI situated at minimum distance from the various diagonals D or bisecting lines.

In the case of a contour 4 that is rectangular in shape, the point PI corresponds to an intersection of the bisecting lines of the corner of the aperture 5, representing the edge of the inserted piece 6 whose adjacent edges rest on the cylindrical body 2 of the endoscope 1.

In accordance with the second method of creation shown in FIGS. 5 and 8, the invention may provide, in the case of a circular contour 4, determination of the point PI, CΔ remaining fixed in the various shots and corresponding to the projection of the central or rotating axis Δ. This consists of performing a substantially 360° rotation of the endoscope or similar device 1 in relation to the opening 5 or cut-out determining the contour 4, and determining the center of the virtual circumference of the circle containing all the circular images resulting from the various shots, and with which these images are locally tangential.

In practice, the body 2 of the endoscope 1 can be rotated in the circular tube 6 and the discoidal area determined that encloses the various “windows” defined by the aperture 5 in the various rotating angular positions (see FIG. 8). The center of this discoidal surface corresponds to point CΔ.

Naturally, the software and hardware used to perform the aforementioned processing and calculations will be known to a person well-versed in the art and do not require an additional description. They can be incorporated into the imaging system used.

The invention also concerns an investigation procedure and/or mini-invasive surgical intervention that, on the one hand, uses a rigid endoscope or similar photographic device 1 consisting of a rigid body 2 inside a cylindrical outer casing 2′ profiled in the direction of the optical axis Σ, or consisting of at least one segment with a rigid end of such casing, fitted with a camera 3 and, on the other hand, a system for acquiring 3D medical images (not shown), both incorporating the area of interest ZI in their respective fields of acquisition. A segment at the end of the endoscope or similar device 1 is visible in the 3D images, thus making it possible to establish a match between the references of camera 3 of the endoscope and the references of the 3D image acquisition system, by determining the orientation of the median axis Δ of the endoscope or similar device and the position of its optical center in the reconstructed 3D images.

This process is characterized, first and foremost, by determining at least a few of the endoscope's settings or those of the similar device 1, especially the offset or misalignment between its optical axis Σ and its central or rotating axis Δ, at least at its end segment, by implementing the procedure described above.

Thanks to this preliminary measure, performed automatically, it is possible to compensate for the misalignment or offsetting between the physical axis Δ of the endoscope 1 and its optical axis Σ, and more generally in relation to the camera 3 (for example one of the CCD type).

According to an advantageous characteristic, the invention may further consist, also in advance, of acquiring successive shots with different orientations of a checkerboard pattern by means of the camera 3 on the endoscope 1, then using these various views to determine the focal distance, especially for calculating the field of vision for a virtual camera, the optical center CΣ in the image plane of camera 3 of the endoscope 1 and the distortion of the lens 7 of the said camera 3, and finally taking into account these intrinsic settings in order to perform a prior calibration of camera 3 and/or subsequent compensation while taking pictures using the endoscope or similar device 1.

The method used in practice for determining the intrinsic settings for camera 3 of the endoscope could, for example, be the one described in the document entitled “A flexible new technique for camera calibration”, Zhang Z., IEEE, Transaction on Pattern Analysis and Machine Intelligence 22, 1330-1334, 2000.

Furthermore, an accelerometer could be mounted at the end of the body 2 of the endoscope 1 to be used for measuring the angular position (pitching and rolling) of the end segment.

In practice, the procedure can consist, during an investigation and/or intervention, of using the results of prior operations to determine misalignment and the intrinsic settings for performing an adjustment and/or recalibration between the internal images supplied by the camera 3 on the endoscope 1 and any external images supplied by the 3D image acquisition system, especially in terms of position, orientation, focus, distortion, and misalignment, with a view to permitting exact superimposition of the information extracted from the external images, especially a volumetric rendering, on the internal images produced by the camera 3.

Generally, the “virtual” point of view produced by intra-operative 3D images is adopted from the viewfinder of the camera 3 on the endoscope in order to provide an increased endoscopic vision from the 3D data.

Of course, the invention is not restricted to the methods of implementation described and represented in the attached drawings. Modifications remain possible, especially from the point of view of the creation of various elements or through the substitution of technical equivalents, yet without nevertheless exceeding the scope of protection of the invention.

1. Procedure for determining the offset or misalignment between the central or rotating axis and the optical axis of a rigid endoscope or similar camera device, consisting of a rigid body with a cylindrical external casing, profiled in the direction of the optical axis, or consisting of at least one rigid segment end with such a casing,
the procedure comprising taking a series of shots, using a camera or similar sensory device (3) that is part of the endoscope or similar device (1), with a field of vision restricted by a contour (4) that is polygonal, circular, or elliptical in shape, whose positioning in relation to the central or rotating axis (Δ) for each shot is physically defined and specific, with a relative angular rotation between the contour (4) and the endoscope or similar device (1) intervening between two successive shots, and determining a point or a pixel (PI, CΔ) in the images acquired successively whose position remains unchanged between the various shots, this point or pixel (PI, CΔ) corresponding to the projection in the image plane (3) of the central or rotating axis (Δ) of the rigid body (2) of the endoscope (1) or similar device or of the rigid end of the latter.

2. Procedure for determining the contour (4), according to claim 1, wherein, in images acquired successively, it presents with a significant contrast in relation to the scene pictured, for example in terms of different levels of gray, color variations, brightness variation, differences in the degree of color saturation or similar, the said contour (4) being defined by an opening or patch (5) as an insert (6).

3. Procedure for determining according to claim 1, wherein the opening or cut-out (5) that defines the outlines of the contour (4) of the field of vision of the endoscope or similar device (1) is provided by a part (6) temporarily mounted on the endoscope (1) or a similar device, a segment on the end of at least one of these, resting for direct or indirect support on the cylindrical external casing (2′).

4. Procedure for determining according to claim 1, further comprising, before taking a series of shots, inserting a body or tubular part (6), of which the interior section is larger than the external section of the cylindrical casing (2′) and that is advantageously provided with a non-reflective interior surface that is dark in color, at the free end (1′) of the endoscope or similar device (1), in such a way that it rests lengthwise on the cylindrical body (2) of the latter or the segment of its rigid end and exceeds its free end in length (1′) to define a restricted shot window, with a field of vision limited peripherally by a contour (4), and changes the relative angular positioning between the said tubular (6) and cylindrical (2) bodies around the said central or rotating axis (Δ) between two successive shots.

5. Procedure for determination, according to claim 1, wherein, in the case of a contour (4) provided with a polygonal opening or cut-out (5), the determination of the point (PI, CΔ) remains fixed in the various shots, and corresponds to the projection of the central or rotating axis (Δ) in the plane of the camera or similar device (3), consisting of extracting at least one diagonal (D) or bisecting line for each of the scenes shown in the images resulting from successive shots, possibly after these have been processed, and determining at least approximately the common point of intersection (PI) of these various diagonals (D) or bisecting lines.

6. Procedure for determination according to claim 5, further comprising applying digital processing to each of the various successive images, a process that is able to remove at least the corner angles or the majority or totality of the polygonal contour (4) visible in the various images, taken with the various angular orientations of the polygonal aperture (5), to be determined in each image processed, the diagonal (D) or bisecting line the end of which touches the edge of the contour (4) being visible in the image in question, to be superimposed on the various images processed with their diagonal (D) or the respective bisecting line chosen and for the shared intersection point (PI) to be determined, at least approximately, in all of the superimposed diagonals (D) or bissecting lines, for which the displacement between successive images has been mapped.

7. Procedure for determination, according to claim 5, wherein each image acquired consists of successively performing the following processing operations: bilateral filtering designed to eliminate noise while retaining the outlines of the contour (4) visible in the image in question; application of the Canny Edge Detector; application of the Hough Transform; grouping of the clearest segments extracted by direction and location; averaging each group of segments to define the angular corners of each contour (4) visible on the various images acquired and defining a corresponding diagonal (D) or bisecting line; determining the point of intersection (PI, CΔ), at least approximately, of the diagonals (D) or bisecting lines selected in the various images.

8. Procedure for determination, according to claim 6, wherein the intersection point, that is at least approximate (PI, CΔ) of the selected diagonals (D) or bisecting lines on the images processed resulting from the taking of various shots consists of applying the least square method, being defined by calculating the position of the point (PI) located at minimum distance from the various diagonals (D) or bisecting lines.

9. Procedure for determination, according to claim 1, wherein, in the case of a circular contour (4), determination of the point (PI, CΔ) tht remains fixed in the various shots, and that corresponds to the projection of the central or rotating axis (Δ), consists of rotating the endoscope of similar device (1) around 360° in relation to the aperture (5) or cut-out that determines the contour (4), and determining the center of the virtual circumferential circle within which are located all of the circular images resulting from the various shots and with which these images are locally tangential.

10. Procedure for investigation and/or mini-invasive surgical intervention implementing, on the one hand, a rigid endoscope or similar photographic device consisting of a rigid body with a cylindrical outer casing profiled in the direction of the optical axis, or consisting of at least one rigid end segment thus encased, fitted with a camera, and, on the other hand, a system for acquiring 3D medical images, both incorporating an area of interest in their respective fields of acquisition, in a segment at the end of the endoscope or similar device that is visible in the 3D images, and thus making it possible to establish a correspondence between the reference system for the endoscope's camera and the reference system for the acquisition of 3D images, through determining the orientation of the median axis of the endoscope or similar device in reconstructed 3D images,
further comprising, primarily, determining at least certain settings for the endoscope or similar device (1), especially the offset or misalignment between its optical axis (Σ) and its central or rotating axis (Δ), at least at the end segment, thus implementing the process according to claim 1.

11. Procedure according to claim 10, further comprising acquiring successive shots with different orientations, of a checkerboard pattern by using the camera (3) of the endoscope (1), then using the various views to determine the focal distance, especially in the calculation of the field of vision of a virtual camera, the optical center (CΣ) in the camera's image plane (3) and the distortion of the lens of the said camera (3) of the endoscope (1), and finally taking account of the intrinsic settings to produce a calibration prior to the camera (3) and/or subsequent compensation during the shots taken using the endoscope or similar device (1).

12. Procedure according to claim 11, further comprising, during the course of an investigation and/or intervention, using the results of prior operations for determining misalignment and the intrinsic settings for performing a readjustment and/or recalibration between the internal images supplied by the camera (3) of the endoscope (1) and the external images supplied by the 3D image acquisition system, especially in terms of position, orientation, focus, distortion, and misalignment.

13. The procedure of claim 5, wherein the polygonal opening or cut-out is rectangular or square in shape.

14. The procedure of claim 7, wherein the angular corners are squares.

15. Procedure for determining according to claim 2, further comprising, before taking a series of shots, inserting a body or tubular part (6), of which the interior section is larger than the external section of the cylindrical casing (2′) and that is advantageously provided with a non-reflective interior surface that is dark in color, at the free end (1′) of the endoscope or similar device (1), in such a way that it rests lengthwise on the cylindrical body (2) of the latter or the segment of its rigid end and exceeds its free end in length (1′) to define a restricted shot window, with a field of vision limited peripherally by a contour (4), and changes the relative angular positioning between the said tubular (6) and cylindrical (2) bodies around the said central or rotating axis (Δ) between two successive shots.

16. Procedure for determining according to claim 3, further comprising, before taking a series of shots, inserting a body or tubular part (6), of which the interior section is larger than the external section of the cylindrical casing (2′) and that is advantageously provided with a non-reflective interior surface that is dark in color, at the free end (1′) of the endoscope or similar device (1), in such a way that it rests lengthwise on the cylindrical body (2) of the latter or the segment of its rigid end and exceeds its free end in length (1′) to define a restricted shot window, with a field of vision limited peripherally by a contour (4), and changes the relative angular positioning between the said tubular (6) and cylindrical (2) bodies around the said central or rotating axis (Δ) between two successive shots.

17. Procedure for determination, according to claim 2, wherein, in the case of a contour (4) provided with a polygonal opening or cut-out (5), the determination of the point (PI, CΔ) remains fixed in the various shots, and corresponds to the projection of the central or rotating axis (Δ) in the plane of the camera or similar device (3), consisting of extracting at least one diagonal (D) or bisecting line for each of the scenes shown in the images resulting from successive shots, possibly after these have been processed, and determining at least approximately the common point of intersection (PI) of these various diagonals (D) or bisecting lines.

18. Procedure for determination, according to claim 3, wherein, in the case of a contour (4) provided with a polygonal opening or cut-out (5), the determination of the point (PI, CΔ) remains fixed in the various shots, and corresponds to the projection of the central or rotating axis (Δ) in the plane of the camera or similar device (3), consisting of extracting at least one diagonal (D) or bisecting line for each of the scenes shown in the images resulting from successive shots, possibly after these have been processed, and determining at least approximately the common point of intersection (PI) of these various diagonals (D) or bisecting lines.

19. Procedure for determination, according to claim 4, wherein, in the case of a contour (4) provided with a polygonal opening or cut-out (5), the determination of the point (PI, CΔ) remains fixed in the various shots, and corresponds to the projection of the central or rotating axis (Δ) in the plane of the camera or similar device (3), consisting of extracting at least one diagonal (D) or bisecting line for each of the scenes shown in the images resulting from successive shots, possibly after these have been processed, and determining at least approximately the common point of intersection (PI) of these various diagonals (D) or bisecting lines.

20. Procedure for determination, according to claim 6, wherein each image acquired consists of successively performing the following processing operations: bilateral filtering designed to eliminate noise while retaining the outlines of the contour (4) visible in the image in question; application of the Canny Edge Detector; application of the Hough Transform; grouping of the clearest segments extracted by direction and location; averaging each group of segments to define the angular corners of each contour (4) visible on the various images acquired and defining a corresponding diagonal (D) or bisecting line; determining the point of intersection (PI, CΔ), at least approximately, of the diagonals (D) or bisecting lines selected in the various images.
